(19) United States
(12) Patent Application Publication
(10) Pub. No.: US 2010/0323407 A1
(43) Pub. Date: Dec. 23, 2010

(54) Manufacturing method of separating and purifying neoagarooligosaccharides having degrees of polymerization from 2 to 22

(75) Inventors: Chorng-Liang Pan, Keelung City (TW); Bartosz Kazlowski, Keelung City (TW); Yuan-Tih Ko, Keelung City (TW); Zwe-Ling Kong, Keelung City (TW); Shao-Chi Wu, Hualien City (TW); Fu-Jin Wang, Danshui Township (TW)

Correspondence Address: BACON & THOMAS, PLLC, 625 SLATERS LANE, FOURTH FLOOR, ALEXANDRIA, VA, 22314-1176, US

(73) Assignee: National Taiwan Ocean University, Keelung City (TW)

(21) Appl. No.: 12/457,661

(22) Filed: Jun. 18, 2009

(51) Int. Cl. C12P 19/04

(52) U.S. Cl. 435/101

(57) ABSTRACT

A manufacturing method of separating and purifying neoagarooligosaccharides having degrees of polymerization of 2-22 includes the steps of adding crude enzyme solutions with agarases produced by Pseudomonas vesicularis MA103 and Aeromonas salmonicida MAEF108, respectively, into an agar polysaccharide extract solution, and obtaining a neoagarooligosaccharides solution after a hydrolysis on the algal polysaccharide (such as agar) is performed; performing a separation to the neoagarooligosaccharides solution by a ultrafiltration (UF) system to obtain a neoagarooligosaccharide eluent; performing a separation to the neoagarooligosaccharide eluent by a semi-preparative high-performance liquid chromatography system equipped with a molecule size exclusion chromatography function to obtain the neoagarooligosaccharides having the degrees of polymerization from 2 to 22; and using a fraction collector to collect the neoagarooligosaccharides, and obtaining purified single neoagarooligosaccharide products with different molecular masses after a freeze-drying process is performed to the neoagarooligosaccharides.

### FIELD OF THE INVENTION

The present invention relates to a method of manufacturing neoagarooligosaccharide (NAOS), in particular to a manufacturing method of separating neoagarooligosaccharides having degrees of polymerization (DP) of 2-22 from agar polysaccharide extracted from Gracilaria (or Gracilaria sp.) to obtain pure single neoagarooligosaccharides with different molecular masses.

### BACKGROUND OF THE INVENTION

As oligosacchrides are defined as prebiotics which are substances capable of selectively promoting the growth of probiotics and serving as food, the special structure of oligosacchrides cannot be decomposed and used by digestive enzymes in human body, and thus oligosacchrides can pass through our digestive tract and enter into our large intestine completely, and the oligosacchrides can be fermented and used selectively by probiotics in our intestine. Oligosaccharides are polymers, each being formed by dehydration and condensation of 2 to 10 monosaccharide molecules. In the nature, oligosaccharide, sucrose, maltose, and lactose exist in plants or fruits in an free state or a glycoside form, and physiologically active oligosaccharides (such as fructo-oligosacchrides, isomalto-oligosaccharides and galacto-oligosacchrides, etc.) bind different monosaccharide molecules by using β-glycosidic bonds, and thus they are difficult to be decomposed by α-glycolytic enzymes existing in our digestive tract, and they can pass through our upper digestive tract, completely enter into our large intestine for providing what the probiotics in the intestines need, and then are fermented into an organic acid to supply energy and lower the pH value in the intestines to suppress intestinal bacteria from producing harmful substances in our body and prevent intestinal microbiota from decomposing bile acid to produce cancer-causing substances. Obviously, oligosaccharides play an important role of maintaining our health and prevent diseases. However, neoagarooligosaccharide related researches showed that the prior art generally uses a high-priced and high-purity agarose as a decomposing substrate for agarase. According to the market price quoted by Sigma Co., the price of agarose is USD 1,330/Kg, and thus the raw material and producton cost for obtaining the extracted oligosacchrides are relatively very high. For example, oligosacchrides (such as neoagarotetraose and neoagarohexaose) cost up to USD 3,125-10,000/g, and are still considered as one of the expensive food materials.

Taiwan is an island surrounded with seas, and has abundant marine resources, and Taiwan is the first country of the world to artificially cultivate Gracilaria (or Gracilaria sp.). The production of Gracilaria in Taiwan has reached 16,775 tons in 2002, and the processed production of Gracilaria has been exported to the overseas markets and brought tremendous economic benefits to Taiwan's marine aquaculture industry. In those years, the small abalone aquaculture is developed rapidly, and the herbivorous small abalones require sufficient and steady supply of algae for their growth. Since Gracilaria has a steady supply and a reasonable price, therefore Gracilaria has replaced other algae gradually and becomes the main alga for the small abalone aquaculture, and this is the main usage of Gracilaria. Up to now, only a small portion of Gracilaria is used as a raw material for producing agar or other food, and thus the Gracilaria is maintained at a low price in the market all the time. It is an important subject for research institutes and marine aquaculture industry to make a good use of the Gracilaria with a low price and a sufficient supply to maximize its values.

In recent years, some research institutes have started using Gracilaria to manufacture oligosacchride by hydrolysis of algal polysaccharide (Such as agar) and whose manufacturing method adopts hot water and an enzyme (such as β-agarase) to process the dried powders of Gracilaria and extract the agar polysaccharide composition therefrom, and use a traditional complicated separation process to separate the oligosaccharides from the agar polysaccharide. The manufacturing method only has a polysacchraide extraction rate of 48.5% and 48.85%. Furthermore, the test of the oligosacchrides rehydrated substances separated from the agar polysaccharide by using the traditional separation process has a reducing-suar content (such as galactose) of 1.27 to 2.36 mg/mL only. Therefore, the efficiency for separating oligosacchrides from Gracilaria by using the traditional method is still very low, and the traditional method cannot maximize its effect.

In view of the shortcomings of the prior art, the inventor of the present invention based on the biochemical properties of Gracilaria to conduct extensive researches and experiments, and finally extracts agar polysaccharides from Gracilaria by using a very simple and low-cost manufacturing process, and separates neoagarooligosaccharides with degrees of polymerization of 2-22 from the agar polysaccharide to obtain pure single neoagarooligosaccharide products of different molecular masses. In addition, the Gracilaria comes with a low price and a sufficient supply, so that the production cost of oligosaccharides can be lowered greatly, and the Gracilaria is able to be a new material for producing the low-cost health food of oligosaccharides of different degrees of polymerization and being added into various different health food sold in the market to maintain human health and achieve the effect of preventing diseases. In the meantime, the Gracilaria also brings a brand new business opportunity.

### SUMMARY OF THE INVENTION

Therefore, it is a primary objective of the present invention to provide a manufacturing method of separating and purifying neoagarooligosaccharides having degrees of polymerization (DP) from 2 to 22, and the method uses a cheap and common agar polysaccharide extract composed of agar polysaccharide with an average molecular mass of 200 kilo-daltons (KDal), wherein one Dal represents a molecular mass of hydrogen atoms. And the polysaccharide hydeolysis reaction is performed by adding crude enzyme solutions of two groups of agar polysaccharide agarases produced by a Pseudomonas (P.) vesicularis MA103 and an Aeromonas (A.) salmonicida MAEF108, stepwisely, into the agar polysaccharide extract to obtain a hydrolyzed neoagarooligosaccharides (NAOS) solution, and whose hydrolysis efficiency is over 10%, and then a ultrafiltration (UF) system performs a separation process to the neoagarooligosaccharides (NAOS) solution to obtain a neoagarooligosaccharide eluent with a molecular mass smaller than 5 KDal. Finally, a semi-preparative high-performance liquid chromatography (HPLC) system equipped with a molecule size exclusion chromatography (SEC) function performs a separation process for the neoagarooligosaccharide eluent to obtain neoagarooligosaccharides with degrees of polymerization (DP) of 2-22, and then a fraction collector is used for the collections to obtain purified single neoagarooligosaccharide products with different molecular masses after a freeze-drying process takes place. The result shows that the method of the present invention can produce various purified neoagarooligosaccharide products in mass production more quickly and efficiently with a lower production cost.

Another objective of the present invention is to use the crude enzyme solutions of agarases produced by the P. vesicularis MA103 and the A. salmonicida MAEF108 having an agarase activity of 250 unit/mL and 278 unit/mL, respectively, and each in an amount of 10 mL to hydrolyze 1,000 mL of agar polysaccharide extract liquid containing Gracilaria dried powder with a content of 0.5% by weight, wherein the crude enzyme solutions with the agarases produced by the P. vesicularis MA103 is added for the hydrolysis at 40° C. for 24 hours, and then the crude enzyme solution with the agarases produced by the A. salmonicida MAEF108 is added for the following hydrolysis also at 40° C. for 24 hours to obtain a neoagarooligosaccharides solution after the hydrolysis, and the total hydrolysis efficiency is over 10%, and then a ultrafiltration (UF) system performs a separation process to the neoagarooligosaccharides solution to obtain a neoagarooligosaccharide eluent with a molecular mass smaller than 5 KDal.

### BRIEF DESCRIPTION OF THE DRAWINGS

FIG. 1 is a flow chart of a manufacturing method of the present invention;

FIG. 2 shows liquid chromatography spectrums obtained through a high-performance liquid chromatography (HPLC) system by analyzing the neoagarooligosaccharides separated from the neoagarooligosaccharide rehydrated solutions, which are obtained by hydrolyzing an agar polysaccharide extract liquid by using crude enzyme solutions with agarases with different units such as (a) 3 unit/mg, (b) 4 unit/mg, (c) 5 unit/mg, and (d) 6 unit/mg in accordance with the present invention;

FIG. 3 shows a group of retention time detected by injecting each separated and purified single neoagarooligosaccharide into a high-performance liquid chromatography (HPLC) system in accordance with the present invention;

FIG. 4 shows a ¹H NMR spectrum obtained by using a nuclear magnetic resonance (NMR) spectroscopy to analyze a main composition N4 of a separated and purified single neoagarooligosaccharide of DP=4; and

FIG. 5 shows negative ion electrospray mass spectrometries of separated and purified single neoagarooligosaccharides of DP=2-12 (as shown in FIGS. 5A-5F) and two standard neoagarooligosaccharides N2 and N6 available in the market (as shown in FIG. 5G) in accordance with the present invention.

### DETAILED DESCRIPTION OF THE PREFERRED EMBODIMENTS

Since genome sequences of an Aeromonas salmonicida MAEF108 and a Pseudomonas vesicularis MA103 have been completed identified in the files, and it is confirmed that the related enzymes (such as agarase having 16 genes, alginate lyase having 5 genes, amylase having 20 genes, cellulase having 2 genes, xylanase having 7 genes) capable of hydrolyzing algal polysaccharides may exist in the crude enzyme solutions of the A. salmonicida MAEF108 and the P. vesicularis MA103, therefore, the present invention utilizes the superior hydrolysis property of the crude enzyme solutions of the two crude enzymes solutions produced by the A. salmonicida MAEF108 and the P. vesicularis MA103 to hydrolyze a low-priced and common agar polysaccharide extract, and the process of preparing the two special agarases contained crude enzyme solutions in accordance with the present invention is described as follows:

(1) In the process of preparing the agarases contained crude enzyme solution produced by the A. salmonicida MAEF108, the A. salmonicida MAEF108 strain is put into a 5-liter (L) fermentation jar containing 3.0 L of MMB-MAEF108 (modified marine broth for the MAEF108 strain, pH 6.0) culture medium with 1.0% of a bacteria inoculum of A. salmonicida MAEF108 activated strain, and the pH value is controlled at 6.2 by using 3.0 N of hydrochloric acid (HCl) and 3.0 N of sodium hydroxide (NaOH) at a temperature of 26° C., and the air flow is maintained at 3.0 L/minute, and the solution is blended by a spin speed of 150 rpm for 48 hours, and the fermented bacteria liquid is collected after being centrifuged by 14,300×g (gravity) at a temperature of 4° C. for 30 minutes, and an upper-layer liquid is collected to obtain a crude enzyme solution of the agarase produced by the A. salmonicida MAEF108, which has an agarase activity of 278 active unit/mL.

(2) In the process of preparing the agarase produced by the P. vesicularis MA103, a P. vesicularis MA103 strain is put into a 5-liter fermentation jar containing 3.0 L of MMB-MA103 (modified marine broth for the strain MA103, pH 6.2) culture medium with 1.0% of a bacteria inoculum of P. vesicularis MA103 activated strain, and controls the pH value at 6.2 by using 3.0 N of hydrochloric acid (HCl) and 3.0 N of sodium hydroxide (NaOH) at a temperature of 26° C., and the air flow is maintained at 4.0 L/minute, and the air is filtered by a membrane filter having pore sizes with a diameter of 0.2 μm, and the solution is blended by a spin speed of 150 rpm for 48 hours, and the fermented bacteria liquid is collected after being centrifuged by 14,300×g (gravity) at a temperature of 4° C. for 30 minutes, and an upper-layer liquid is collected to obtain a crude enzyme solution with the agarases produced by the P. vesicularis MA103, which has an agarase activity of 250 active unit/mL.

The agar polysaccharide extract used in the present invention is made of a low-priced and common Gracilaria (or Gracilaria sp.) by grounding the dried Gracilaria into powder by a grounding machine, and sieving the powder by a standard sieve having meshes with a diameter of 297 μm (48 mesh), and storing the Gracilaria dried powder into a freezer at −20° C. for future use. Mix 500 g of Gracilaria dried powder in pure water uniformly, wherein the ratio of Gracilaria dried powder and pure water by weight is 1:50. After the two are mixed uniformly, the aqueous solution is heated for 40 minutes at a temperature of 121° C. (in a autoclave), and an upper-layer liquid is collected to obtain the agar polysaccharide extract liquid. A freeze-dryer at a temperature below −18° C. is used for freeze-drying the agar polysaccharide extract liquid to produce 289 g of freeze-dried polysaccharides extract in lyophilized powder. Now, the main composition of agar polysaccharide extract powder is an agar polysaccharide with an average molecular mass of 200 KDal.

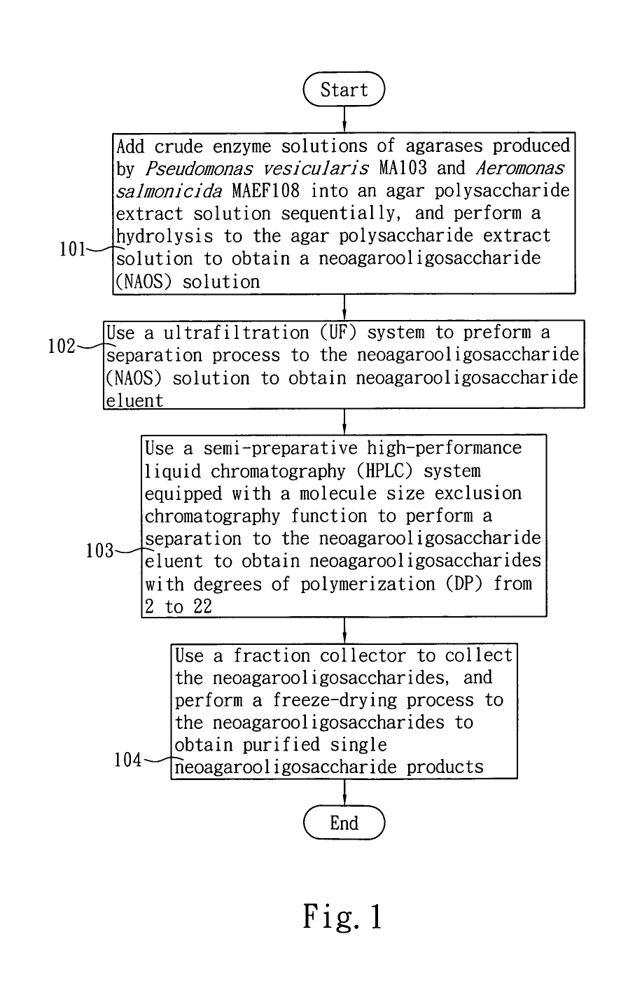

With reference to FIG. 1 for a flow chart of a manufacturing method of the present invention, the method comprises the following steps to produce purified single neoagarooligosaccharide products of different molecular masses quickly by a low production cost and improve the production efficiency of the neoagarooligosaccharide:

Step (101): Put 289 g of agar polysaccharide extract powder into a 5-liter fermentation jar, and use 3.0 liters of buffer solution to recover the agar polysaccharide extract powder into the agar polysaccharide extract liquid, wherein the reactant concentration of the Gracilaria is 0.5%, and the proportion of the mixture of the buffer solution (Tris-HCl buffer, pH 6.2), sodium chloride (NaCl) and calcium chloride (CaCl₂) by weight is 0.5:1.0:0.1, and the pH value is a fixed at pH 6.0, and the additive for growing agarases is 0.3% of agar by weight. Then, add crude enzyme solutions of agarases produced by a P. vesicularis MA103 and an A. salmonicida MAEF108 into the agar polysaccharide extract solution, wherein cultures of MA103 and MAEF108 have an agarase activity of 250 active unit/mL and 278 active unit/mL, respectively, and the ratio of MA103 or MAEF108 to the agar polysaccharide extract liquid is 10 mL:1,000 mL, such that a hydrolysis is performed to agar polysaccharide extract solution under the conditions that the blending is held at a spin speed of 150 rpm, and the reaction temperature is 40° C., and the crude enzyme solution of the agarase produced by the P. vesicularis MA103 is added and reacted for 24 hours, and then the crude enzyme solution of the agarase produced by the A. salmonicida MAEF108 is added and reacted for 24 hour. After the two-stage hydrolysis is completed, a neoagarooligosaccharides (NAOS) solution is obtained.

To know about the hydrolysis efficiency from algal polysaccharide (such as agar) to the neoagarooligosaccharides solution, the inventor of the present invention performs a concentration process to the neoagarooligosaccharides solution, and further performs a gel-permeation chromatography to the concentrated solution of the neoagarooligosaccharides solution, wherein tubes each having a height of 100 cm and a diameter of 2.6 cm are used for containing Sephadex G-10 (Brand: Pharmacia, USA), and sterile water is used as an eluent, and the gel-permeation chromatography of the Sephadex G-10 shows that the total sugar content (A_{480 nm}) of the collected liquid separated by each tube contains oligosacchrides with a molecular mass from 163.1 to 1,213.9 Dal, and the production quantity of various single neoagarooligosaccharide (NAOS) products with different molecular masses such as monosaccharides (galactose and/or 3,6-anhydro-L-galactose), disaccharide (neoagarobiose), tetraose (neoagarotetraose), and hexaose (neoagarohexaose) are 2.36 g, 16.76 g, 13.18 g and 20.88 g, respectively (as shwon in Table 1), and whose production rates are 0.47%, 3.35%, 2.64%, and 4.18%, respectively. In other words, the hydrolysis efficiency to produce NAOSs (DP=2-6) is over 10%.

TABLE 1
Production Rate of Oligosacchrides of Gracilaria

| Procedure | Composition | Production Rate (%) | |
|---|---|---|---|
| Raw Material | Gracilaria dried powder | 500 | g |
| Extraction | Agar polysaccharides extracted | 289 | g |
| | powder | | |
| Hydrolysis | hydrolyzed solution of agar | 5.78 | L |
| | polysaccharides extract | | |
| Concentration | Concentrated solution of the | 1,200 | mL |
| Treatment | neoagarooligosaccharides | | |
| | rehydrated solution | | |
| Gel-permeation | Monosaccharides (Galactose | 2.36 | g (0.47%) |
| Chromatography | and/or 3,6-anhydro-L-galactose) | | |
| | Biose (Neoagarobiose) | 16.76 | g (3.35%) |
| | Tetraose (Neoagarotetraose) | 13.18 | g (2.64%) |
| | Hexaose (Neoagarohexaose) | 20.88 | g (4.18%) |
| | Total | 53.18 | g (10.64%) |

Step (102): Use a ultrafiltration (UF) system (a disposable modular tangential flow system) and a 5 Kdal molecular weight cut-off (MWCO) polyethersulfone membrane to preform a separation process and then through a vacuum concentration process to obtain 5.78 L and then 289 mL NAOSs solutions, respectively, with a molecular mass smaller than 5 Kdal.

Step (103): Use a semi-preparative high-performance liquid chromatography (HPLC) system equipped with a molecule size exclusion chromatography (column) function to perform a separation to the neoagarooligosaccharides eluent so as to obtain different single neoagarooligosaccharides with degrees of polymerization (DP) of 2, 4, 6, 8, 10, 12, 14, 16, 18, 20, and 22 at different retention time (about 15-30 minutes), respectively.

Step (104): Use a fraction collector to collect the neoagarooligosaccharides with different degrees of polymerization by the semi-preparative high-performance liquid chromatography (HPLC) system, so as to obtain purified single neoagarooligosaccharide products of different molecular masses and then the freeze-drying process is performed to harvest each of these neoagarooligosaccharides individually.

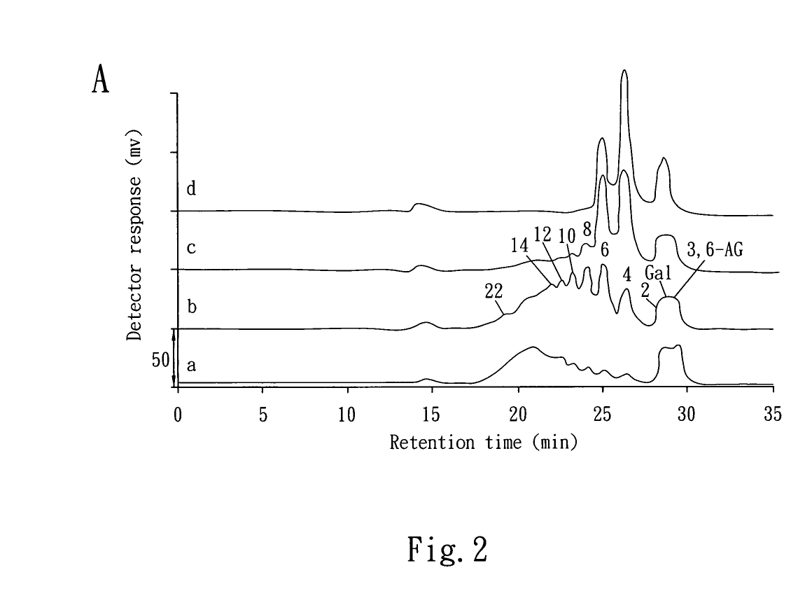

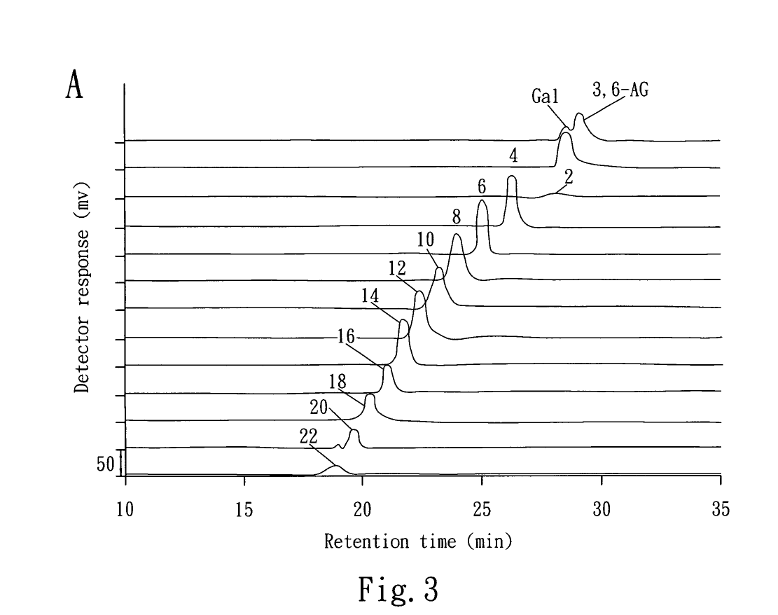

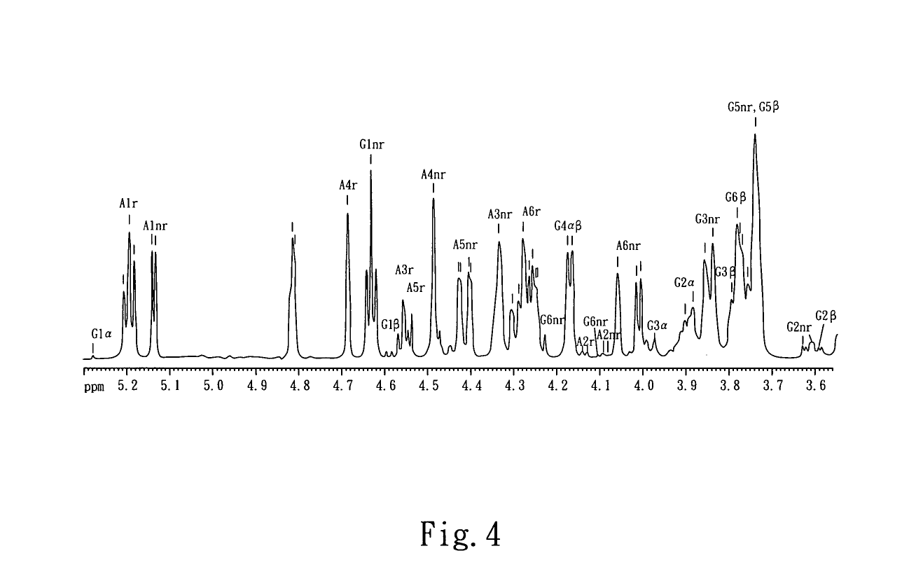

The present invention uses chromatograms of the high-performance liquid chromatography (HPLC) system to analyze the neoagarooligosaccharides separated from the neoagarooligosaccharide hydrolyzed solutions, which are obtained by hydrolyzing an agar polysaccharide extract solution by using crude enzyme solutions with agarases with different units such as (a) 3 unit/mg, (b) 4 unit/mg, (c) 5 unit/mg, and (d) 6 unit/mg, in other experimetns conducted by the inventor, and proves that, as shown in FIG. 2, the present invention is able to use the semi-preparative high-performance liquid chromatography (HPLC) system to separate and collect the neoagarooligosaccharides with degrees of polymerization of 2-22 from the neoagarooligosaccharide rehydrated solution, and then freeze-dry the neoagarooligosaccharides to obtain pure single neoagarooligosaccharide products. In FIG. 3, the degrees of polymerization are estimated according to the standard curve of retention time versus molecular mass measured by the high-performance liquid chromatography (HPLC) system after the collected liquid of separated and purified single neoagarooligosaccharides are injected into the high-performance liquid chromatography (HPLC) system. FIG. 3 shows a group of different retention time detected by the high-performance liquid chromatography (HPLC) system after the separated and purified single neoagarooligosaccharide are re-injected into the high-performance liquid chromatography (HPLC) system in accordance with the present invention. FIG. 4 shows a ¹H NMR spectrum obtained by using a nuclear magnetic resonance (NMR) spectroscopy to analyze a main composition N4, a separated and purified single neoagarooligosaccharide of DP=4, wherein a peak label A represents 3,6-anhydro-L-galactose, a peak label G represents galactose, nr and r represent non-reducing and reducing ends, respectively, a and b represent positoins of protons of the reducing end, and numbers 1 to 6 prepresent proton places.

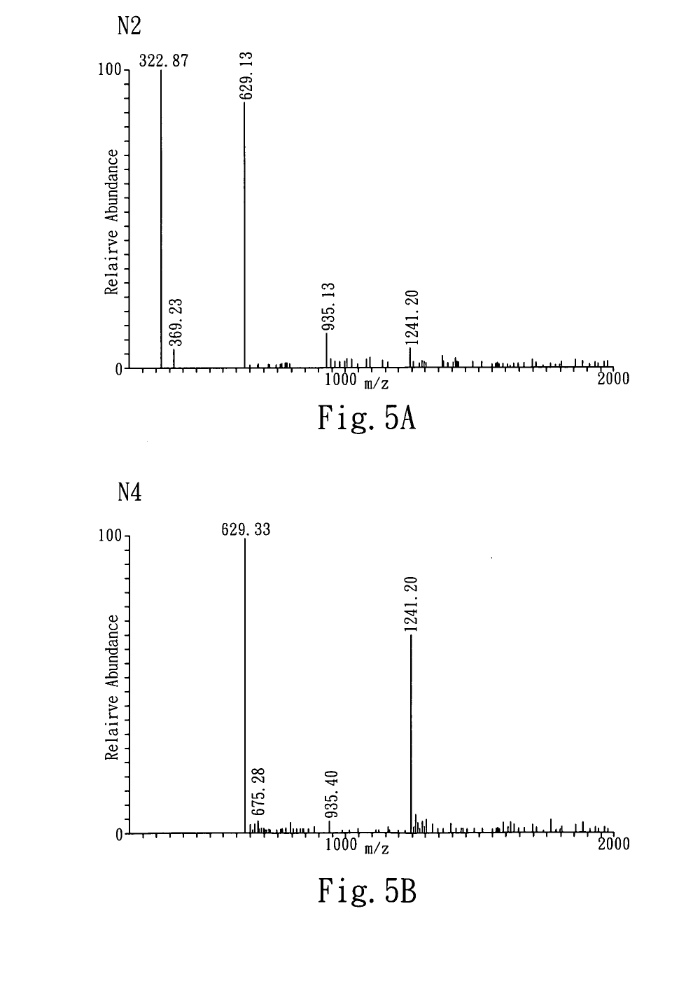

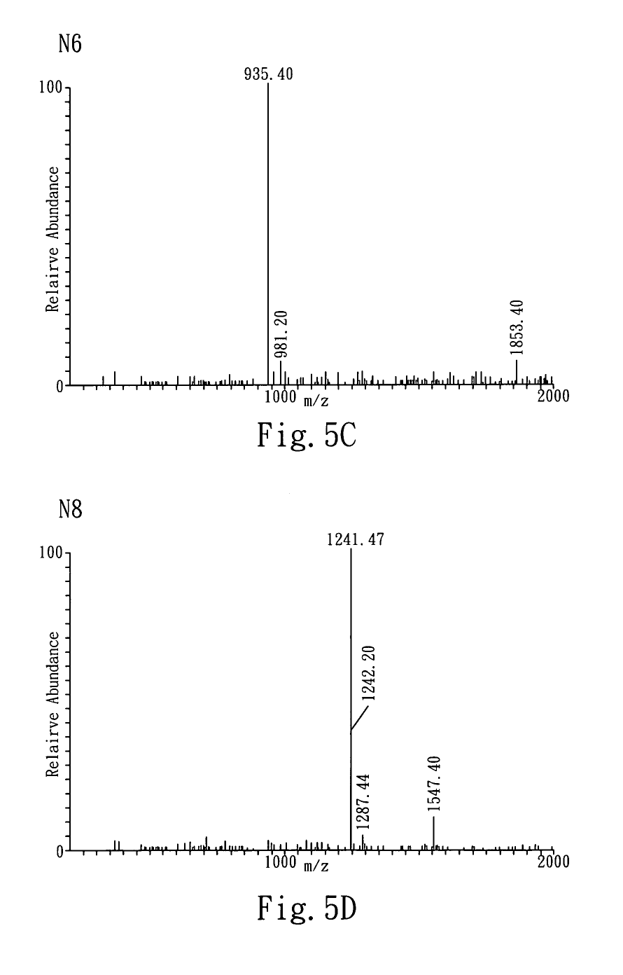

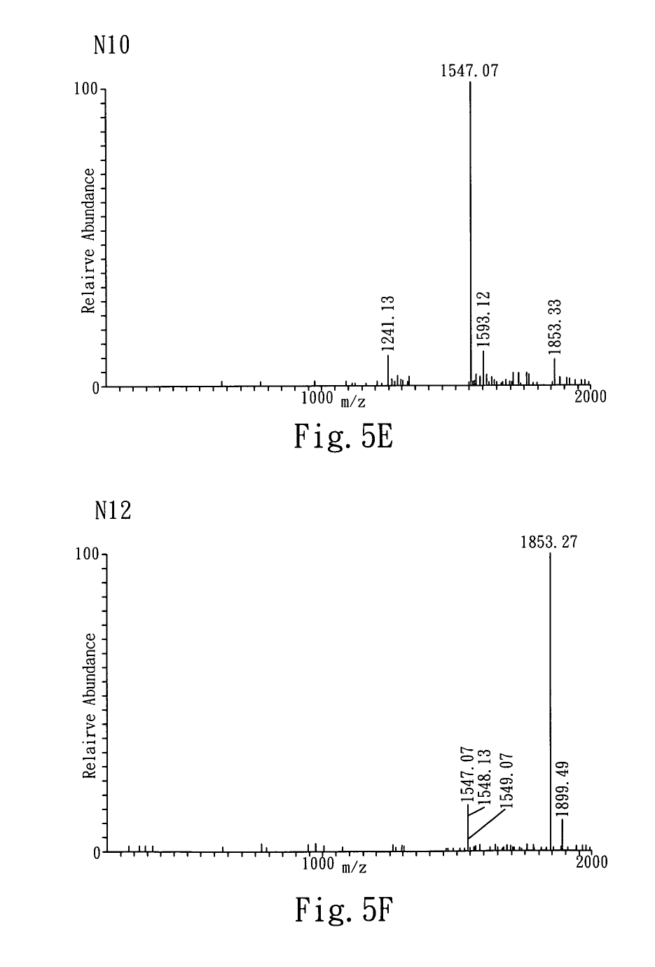

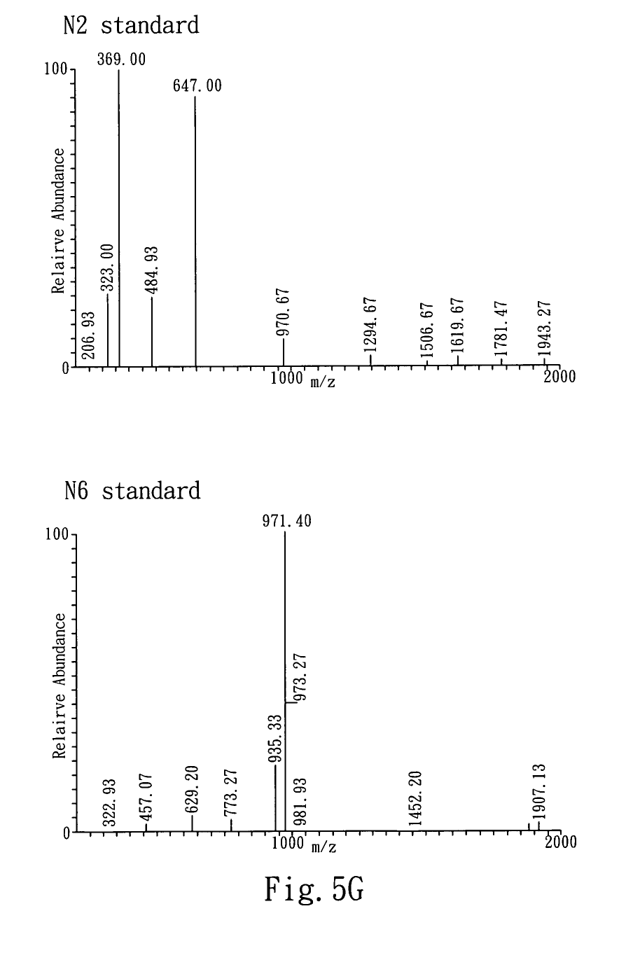

FIG. 5 shows negative ion electrospray mass spectrometries (ESIMS) of separated and purified single neoagarooligosaccharides of DP=2-12 (as shown in FIGS. 5A-5F) and two standard neoagarooligosaccharides N2 and N6 available in the market (as shown in FIG. 5G) in accordance with the present invention.

Since the manufacturing process of the present invention adotps 500 g of Gracilaria dried powder which only costs USD 20-25/Kg to maufacture approximately 53.18 g of neoagarooligosaccharides with degrees of polymerization of 2-22, and the production efficiency is over 10%, and its value as quoted by Sigma Co. Catalogue is apprixlately USD 3,300/g. Thus, the present invention is able to fully use the low-price and common Gracilaria as the raw material, and use the crude enzyme solutions of P. vesicularis MA 103 and A. salmonicida MAEF 108 to perform hydrolysis to the agar polysaccharide extract, and then uses a ultrafiltration system to perform a separation to the neoagarooligosaccharides solution to obtain a neoagarooligosaccharides eluent with a molecular mass smaller than 5 Kdal, and finally uses a semi-preparative high-performance liquid chromatography (HPLC) system equipped with a molecule size exclusion chromatography (SEC) fucntion to perform a separation to the neoagarooligosaccharide eluent to quickly produce high-priced and purified neoagarooligosaccharide products with degrees of polymerization of 2, 4, 6, 8, 10, 12, 14, 16, 18, 20, and 22, respectively. In summation of the description above, the present invention can mass produce high-priced and purified neoagarooligosaccharide products by a simple and quick manufacturing process and a low production cost.

While the invention herein disclosed has been described by means of specific embodiments, numerous modifications and variations could be made thereto by those skilled in the art without departing from the scope and spirit of the invention set forth in the claims.

What is claimed is:

1. A manufacturing method of separating and purifying neoagarooligosaccharides having degrees of polymerization from 2 to 22, comprising:
sequentially adding crude enzyme solutions of agarases produced by a Pseudomonas vesicularis MA103 and an Aeromonas salmonicida MAEF108 into an agar polysaccharide extract solution, and obtaining a neoagarooligosaccharides solution after a hydrolysis is performed;
performing a separation to the neoagarooligosaccharides solution by using a ultrafiltration (UF) system to obtain a neoagarooligosaccharide eluent;
performing a separation of the neoagarooligosaccharide eluent by using a semi-preparative high-performance liquid chromatography (HPLC) system equipped with a molecule size exclusion chromatography function to obtain the neoagarooligosaccharides having the degrees of polymerization from 2 to 22; and
using a fraction collector to collect the neoagarooligosaccharides, and obtaining purified single neoagarooligosaccharide products with different molecular masses after a freeze-drying process is performed to the neoagarooligosaccharides.

2. The method of claim 1, wherein the agar polysaccharide extract liquid is mainly composed of agar polysaccharide with an average molecular mass of 200 KDal and pure water.

3. The method of claim 2, wherein the neoagarooligosaccharide eluent is mainly composed of a mixed solution of neoagarooligosaccharides with a molecular mass smaller than 5 KDal.

4. The method of claim 3, further comprising: putting a dried powder of the agar polysaccharide extract into a fermentation tank, and using a buffer solution to recover the dried powder into the agar polysaccharide extract liquid, such that the Gracilaria has a reactant concentration of 0.5%.

5. The method of claim 4, wherein the crude enzyme solution with the agarases produced by the P. vesicularis MA103 has an agarase activity of 250 active unit/mL, and the crude enzyme solution with the agarases produced by the A. salmonicida MAEF108 has an agarase activity of 278 active unit/mL, and the crude enzyme solution of the agarases produced by the P. vesicularis MA103 or A. salmonicida MAEF108 and the agar polysaccharide extract liquid are mixed in a ration of 10 mL:1000 mL.

6. The method of claim 5, wherein the crude enzyme solution of the agarases produced by the P. vesicularis MA103 is added in the agar polysaccharide extract liquid for the hydrolysis for at least 24 hours at a reaction temperature of 40 deg. C., and then the crude enzymes solution with the agarases produced by the A. salmonicida MAEF108 is added for the hydrolysis for at least 24 hours at a reaction temperature of 40 deg. C.
